(19) United States
(12) Patent Application Publication
(10) Pub. No.: US 2022/0387472 A1
(43) Pub. Date: Dec. 8, 2022

(54) COMPOSITIONS AND METHODS FOR TREATING CANCER

(71) Applicant: AIM ImmunoTech Inc., Ocala, FL (US)

(72) Inventors: Thomas K. EQUELS, Ocala, FL (US); David R. STRAYER, Bryn Mawr, PA (US)

(73) Assignee: AIM ImmunoTech Inc., Ocala, FL (US)

(21) Appl. No.: 17/770,158

(22) PCT Filed: Sep. 21, 2021
(86) PCT No.: PCT/US2021/051369

§ 371 Date: Apr. 19, 2022

Related U.S. Application Data
(60) Provisional application No. 63/081,296, filed on Sep. 21, 2020.

(51) Int. Cl. A61K 31/713; A61K 31/519; A61K 31/513; A61K 31/4745; A61K 31/282; A61P 35/00; A61P 1/18; A61K 31/7068

(52) U.S. Cl. CPC A61K 31/713; A61K 31/519; A61K 31/513; A61K 31/4745; A61K 31/282; A61P 35/00; A61P 1/18; A61K 31/7068

(57) ABSTRACT

Disclosed is a method for treating a cancer, such as pancreatic cancer, in a subject. The method comprises a first step of administering to the subject a standard of care therapy such as FOLFIRINOX and administering to the subject a therapeutic double stranded RNA (tdsRNA) which can be Rintatolimod.

### PRIORITY

This application claims the benefit of priority to U.S. Provisional Patent Application No. 63/081,296 filed Sep. 21, 2020, the entire content of which is incorporated herein by reference.

### BACKGROUND

Pancreatic cancer, or carcinoma of the pancreas, is a disease that is hard to detect and is usually not diagnosed until it is in a late metastasis stage (stage 4). This, in turn, leads to a high mortality rate and poor treatment response. Pancreatic cancer has a 5 year survival rates of just 6% making it the fourth leading cause of cancer death in the United States—one of the lowest survival rates among the common cancers. Further, the pancreatic cancer incidence rate is increasing and it is anticipated to move from the fourth to the second leading cause of cancer death in the United States by 2030. Thus, treatment of this cancer has become a significant public health concern.

### BRIEF DESCRIPTION

One embodiment is directed to a method for treating a cancer in a subject in need thereof. The method comprises a first step of administering to the subject a cancer therapy which is a standard of care for the cancer; and a second step of administering to the subject at least an effective amount of a therapeutic double stranded RNA (tdsRNA). The first step and the second step may be performed in any order or simultaneously.

Another embodiment is directed to a method for treating a cancer in a subject in need thereof, the method comprising administering to the subject a compound comprising an effective amount of a therapeutic double stranded RNA (tdsRNA).

In any embodiment, the cancer may be pancreatic cancer. For example, the cancer may be at least one selected from the group consisting of: pancreatic carcinoma; advanced pancreatic carcinoma; locally advanced pancreatic cancer (LAPC); metastasized pancreatic cancer; and pancreatic cancer metastasized after resection.

In any embodiment, the standard of care for a cancer may be a FOLFIRINOX treatment regimen. It follows that, in this case, the first step of the method may be administering to the subject the FOLFIRINOX treatment regimen or at least one round of FOLFIRINOX. In any embodiment, the standard of care for a cancer may be a Gemcitabine treatment regimen. It follows that, in this case, the first step of the method may be administering to the subject the Gemcitabine treatment regimen or at least one round of Gemcitabine. FOLFIRINOX is a combination of drugs, including: FOL—folinic acid (also called leucovorin, calcium folinate, or FA); F—fluorouracil (also called 5FU); IRIN—irinotecan; and OX—oxaliplatin. Therefore, in any embodiment, tdsRNA may be administered at the same time as any one of the components or all of the components of FOLFIRINOX. As another example, the tdsRNA and Gemcitabine may be administered together.

In any embodiment, the tdsRNA may be at least one selected from the group consisting of: rIₙ.r(CₓU)ₙ (formula 1); rIₙ.r(CₓG)ₙ (formula 2); rAₙ.rUₙ (formula 3); rIₙ.rCₙ (formula 4); and rugged dsRNA (formula 5); wherein x is at least one selected from the group consisting of 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 4-29, 4-30, 14-30, 15-30, 11-14, and 30-35.

In any embodiment, at least 90 wt % of the tdsRNA may be larger than a size selected from the group consisting of: 40 basepairs; 50 basepairs; 60 basepairs; 70 basepairs; 80 basepairs; and 380 basepairs. In any embodiment, at least 90 wt % of the tdsRNA may be smaller than a size selected from the group consisting of: 50,000 basepairs; 10,000 basepairs; 9000 basepairs; 8000 basepairs; 7000 basepairs; and 450 basepairs.

In any embodiment, the variable “n” of formula 1-5 may be a number with a value selected from the group consisting of: 40 to 50,000; 40 to 40,000; 50 to 10,000; 60 to 9000; 70 to 8000; 80 to 7000; and 380 to 450.

For example, n may be from 40 to 40,000; the tdsRNA may have about 4 to about 4000 helical turns of duplexed RNA strands; or the tdsRNA has a molecular weight selected from the group consisting of: 2 kDa to 30,000 kDa; 25 kDa to 2500 kDa; and 250 kDa to 320 kDa.

The tdsRNA may comprise, or consist of, or consist essentially of rIₙ.r(C₁₁₋₁₄U)ₙ; and rugged dsRNA.

In any embodiment, the Rugged dsRNA may have: a single strand comprised of r(C₄₋₂₉U)ₙ, r(C₁₁₋₁₄U)ₙ, or r(C₁₂U)ₙ; and an opposite strand comprised of r(I); wherein the single strand and the opposite strand do not base pair the position of the uracil base, and wherein the single strand and the opposite strand are partially hybridized.

In any embodiment, the Rugged dsRNA may have: (1) a molecular weight of about 250 kDa to 500 kDa; (2) wherein each strand of the rugged dsRNA is from about 400 to 800 basepairs in length; or (3) the rugged tdsRNA has about 30 to 100 or 30 to 60 helical turns of duplexed RNA.

In any embodiment, the Rugged dsRNA may be a Rugged dsRNA which is resistant to denaturation under conditions that are able to separate hybridized poly(riboinosinic acid) and poly(ribocytosinic acid) strands (rIₙ.rCn).

In any embodiment, the rugged dsRNA may be an isolated double-stranded ribonucleic acid (dsRNA) active under thermal stress comprising: each strand with a molecular weight of about 250 KDa to about 500 KDa, 400-800 basepairs, or 30 to 60 helical turns of duplex RNA; a single strand comprised of poly(ribocytosinic₄₋₂₉ uracilic acid) and an opposite strand comprised of poly(riboinosinic acid); wherein the two strands do not base pair the position of the uracil base; wherein the two strands base pair the position of the cytosine base; and wherein the strands are partially hybridized.

In any embodiment, the tdsRNA may comprise 0.1-12 mol % rugged dsRNA, preferably the tdsRNA comprises 0.1-5 mol % rugged dsRNA.

In any embodiment, the tdsRNA may comprise at least one pharmaceutically acceptable carrier.

In any embodiment, the administration or administering of the tdsRNA may be performed by at least one method selected from the group consisting of: intravenous administration; systemic administration; parenteral administration; intradermal administration; subcutaneous administration; intramuscular administration; intranasal administration; intranasal and oral administration; intraperitoneal administration; intracranial administration; intravesical administration; oral administration; topical administration; and enteral administration.

In any embodiment, the administration of tdsRNA may be at a dosage of 25 mg to 700 mg of tdsRNA per day or per dose; 20 mg to 200 mg of tdsRNA per day or per dose; 50 mg to 150 mg of tdsRNA per day or per dose; or 80 mg to 140 mg of tdsRNA per day or per dose.

In any embodiment, the administration the tdsRNA is administered at a frequency selected from the group consisting of: one dose per day, one dose every 2 days, one dose every 3 days, one dose every 4 days, one dose every 5 days, one dose a week, two doses a week, three doses a week, one dose every two weeks, one dose every 3 weeks, one dose every 4 weeks, and one dose a month.

One preferred dosage is tdsRNA (e.g., rintatolimod (AMPLIGEN®)) 200 mg twice weekly for 2 weeks, then 400 mg twice weekly thereafter (i.e., after the first two weeks, which is from the third week and thereafter). The treatment may be maintained for a number of weeks such as 12 weeks, 15 weeks, 18 weeks. Alternatively, the treatment may be continuous. Continuous treatment, in this case, refers to 400 mg twice weekly after the initial 2 weeks for as long as needed. Alternatively, the treatment may be 200 mg twice weekly. As another alternative, the treatment may be 400 mg twice weekly. One preferred administration method is intravenous administering.

In any embodiment, the subject may be any animals described in this disclosure. The subject is preferably a mammal, and more preferably a human.

In any embodiment, the tdsRNA may be a tdsRNA combined with a pharmaceutically acceptable carrier.

Cancer, including pancreatic cancer, is most often manifested as a tumor, and the tumor may be a solid or a liquid tumor. Treatment generally relates to increasing the survival of the subject, preventing the growth or spread of the tumor, or reducing or eliminating the tumor. Therefore, in one embodiment, the methods have an effect on the subject, or treats (e.g., treatment, treating) the subject. The effect or “treating” may be at least one selected from the group consisting of: increasing survival of the subject; increasing time of progression of the subject; inhibiting tumor growth; inducing tumor cell death; increasing tumor regression; preventing tumor recurrence; preventing tumor growth; preventing tumor spread; delaying tumor recurrence; delaying tumor growth; delaying tumor spread; and promoting tumor elimination.

All possible combinations and permutations of individual elements, embodiments, and aspects and parts thereof, in this disclosure, are also considered to be aspects and embodiments of the disclosure.

While the invention has been described in connection with what is presently considered to be the most practical and preferred embodiment, it is to be understood that the invention is not to be limited to the disclosed embodiment, but on the contrary, is intended to cover various modifications and equivalent arrangements included within the spirit and scope of the appended claims.

### DETAILED DESCRIPTION

1. Overview of Disclosure

According to the Pancreatic Cancer Action Network, Pancreatic cancer is the fourth leading cause of cancer death in the U.S. It is the only cancer of the most commonly diagnosed with a five-year survival rate at just six percent. Pancreatic cancer is anticipated to move from the fourth to the second leading cause of cancer death in the U.S. by 2020, based on current projections. Accordingly, both the projected number of new pancreatic cancer cases and pancreatic cancer deaths are expected to double by 2030. In the EU, the incidence is continuing to increase and death rate is projected to increase by ˜30% to ˜112,000 new cases per year by 2025 and will exceed the number of breast cancer deaths (J. Ferlay, C. Partensky & F. Bray (2016) More deaths from pancreatic cancer than breast cancer in the EU by 2017, Acta Oncologica, 55:9-10, 1158-1160).

TABLE 1
Projected Pancreatic and Breast Cancer Deaths
in the EU by 2025

| Year | Pancreas Cancer | Breast Cancer |
|---|---|---|
| 2010 | 76,000 | 92,000 |
| 2017 | 91,500 | 91,000 |
| 2025 | 111,500 | 90,000 |

Currently, surgery is the only potentially curative option, but only around 15% of patients are eligible at initial diagnosis since most pancreatic cancers are detected in an advanced stage of the disease. Around 20% of patients are diagnosed with locally advanced pancreatic cancer and the remaining 30-50% present with metastatic disease. It is clear that new treatment options are desperately needed for this devastating malignancy.

In this disclosure, the standard of care for pancreatic cancer should be considered to be Gemcitabine, FOLFIRINOX, or both Gemcitabine and FOLFIRINOX in any order or simultaneously. For several years since its introduction, gemcitabine monotherapy was the standard palliative treatment for this disease. Gemcitabine was approved based on a combined benefit of symptom palliation and survival, though its survival prolongation benefit is modest at best. Frustratingly, attempts to improve on these mediocre benefits of Gemcitabine had been met with little success and no new treatments were established for metastatic pancreatic cancer for several years. Against this background, significant enthusiasm was generated when a phase III randomized clinical trial comparing Gemcitabine with the combination regimen FOLFIRINOX (consisting of the combination of 5-fluorouracil, leucovorin, irinotecan, and oxaliplatin) showed an unprecedented survival benefit. FOLFIRINOX quickly became the new standard of care for patients with metastatic pancreatic cancer who could tolerate it.

2. FOLFIRINOX

FOLFIRINOX is a combination of drugs, including: FOL—folinic acid (also called leucovorin, calcium folinate or FA); F—fluorouracil (also called 5FU); IRIN—irinotecan; and OX— oxaliplatin.

A typical FOLFIRINOX regimen sequence is as follows. On day one: Oxaliplatin 85 mg/m² IV over 2 hours (total dose/cycle, 85 mg/m²); Leucovorin 400 mg/m² IV over 2 hours (total dose/cycle, 400 mg/m²); Irinotecan 180 mg/m² IV over 90 minutes (total dose/cycle, 180 mg/m²); Fluorouracil 400 mg/m² IV bolus; followed by 2,400 mg/m² IV continuous infusion (CI) over 46 hours beginning on Day 1 (total dose/cycle [bolus and CI], 2,800 mg/m²). This cycle which begins on day 1 and is summarized in the following TABLE 2.

TABLE 2
One Possible FOLFIRINOX Regimen

| | | | Given |
|---|---|---|---|
| | | | on |
| Drug | Dose and route | One Possible Administration Method | days |
| Oxaliplatin* | 85 mg/m² IV | Dilute in 500 mL D5W and administer over two | Day 1 |
| | (85 mg per | hours (prior to leucovorin). Shorter oxaliplatin | |
| | square meter | administration schedules (e.g., 1 mg/m² per | |
| | (m²) of body | minute) appear to be safe. | |
| | surface area | | |
| | intravenous) | | |
| Leucovorin | 400 mg/m² IV | Dilute in 250 mL D5W and administer over two | Day 1 |
| | | hours (after oxaliplatin). | |
| Irinotecan | 180 mg/m² IV | Dilute in 500 mL D5W and administer over 90 | Day 1 |
| | | minutes. Administer concurrent with the last 90 | |
| | | minutes of leucovorin infusion, in separate bags, | |
| | | using a Y-line connection. | |
| Fluorouracil | 400 mg/m² IV | Give undiluted (50 mg/mL) as a slow IV push | Day 1 |
| (FU) | bolus | over five minutes (administer immediately after | |
| | | leucovorin). | |
| FU | 2400 mg/m² IV | Dilute in 500 to 1000 mL 0.9% NS or D5W and | Day 1 |
| | | administer as a continuous IV infusion over 46 | |
| | | hours (begin immediately after FU IV bolus). To | |
| | | accommodate an ambulatory pump for outpatient | |
| | | treatment, can be administered undiluted (50 | |
| | | mg/mL) or the total dose diluted in 100 to 150 mL | |
| | | NS. | |

This day one administration, which can last longer than one day but is started on day one, as described above, is repeated every 14 days until disease progression or unacceptable toxicity. A typical FOLFIRINOX regimen is 12 cycles of 14 days. We have found that a treatment regimen comprising FOLFIRINOX and tdsRNA is surprisingly effective for the treatment of cancer such as pancreatic cancer.

3. tdsRNA

This disclosure relates to, inter alia, tdsRNA. tdsRNA can also be called “therapeutic dsRNA,” or “therapeutic double-stranded RNA” and these terms have the same meaning. In this section, or anywhere in this disclosure, a reference to “tdsRNA” would include, at least, a reference to a composition comprising tdsRNA, a medicament comprising tdsRNA, a composition comprising rintatolimod, or a medicament comprising rintatolimod. Further, any reference to tdsRNA would include at least AMPLIGEN® (rintatolimod).

“r” and “ribo” have the same meaning and refer to ribonucleic acid or the nucleotides or nucleosides that are the building block of ribonucleic acid.

RNA consists of a chain of linked units called nucleotides. This disclosure relates mostly to RNA and, therefore, unless otherwise specified, the nucleotides and bases expressed refers to the ribo form of the nucleotide or base (i.e., ribonucleotide with one or more phosphate groups). Therefore “A” refers to rA or adenine, “U” refers to rU or uracil, “C” refers to rC or cytosine, “G” refers to rG or guanine, “I” refers to rI or inosine, “rN” refers to rA, rU, rC, rG or rI. Each of these (i.e., A, U, C, G, I) may have one or more phosphate groups as discussed above depending on whether they are part of a chain (i.e., RNA) or free (nucleoside, nucleotide, etc.).

“n” is a positive number and refers to the length (in bases for single stranded nucleic acid or in basepairs in double stranded nucleic acid) of ssRNA or dsRNA or to the average length of a population of ssRNA or dsRNA. “n” can be a positive integer when referring to one nucleic acid molecule or it can be any positive number when it is an average length of a population of nucleic acid molecules.

An RNA may have a ratio of nucleotides or bases. For example, r(C₁₂U)ₙ denotes a single RNA strand that has, on average 12 C bases or nucleotides for every U base or nucleotide. As another example, r(C₁₁₋₁₄U)ₙ denotes a single RNA strand that has, on average 12 C bases or nucleotides for every U base or nucleotide.

Formulas: As an example, the formula “rIₙ.r(C₁₂U)ₙ” can be expressed as riboIₙ.ribo(C₁₂U)ₙ, rIₙ.ribo(C₁₂U)ₙ, or riboIₙ.r(C₁₂U)ₙ, refers to a double-stranded RNA with two strands. One strand WO is poly ribo-inosine of n bases in length. The other strand is ssRNA of random sequence of C and U bases, the random sequence ssRNA is n bases in length, and a ratio of C bases to U bases in the random sequence ssRNA is about 12 (i.e., mean 12 C to 1 U). The terms “r” and “ribo” have the same meaning in the formulas of the disclosure. Thus, rI, riboI, r(I) and ribo(I) refer to the same chemical which is the ribose form of inosine. Similarly, rC, riboC, r(C) and ribo(C) all refer to cytidine in the ribose form which is a building block of RNA. rU, riboU, r(U) and ribo(U) all refer to Uracil in the ribose form which is a building block of RNA.

The “.” symbol indicates that one strand of the dsRNA is hybridized (hydrogen-bonded) to the second strand of the same dsRNA. Therefore, rIₙ.r(C₁₂U)ₙ is double-stranded RNA comprising two ssRNA. One ssRNA is poly(I) and the other ssRNA is poly(C₁₂U). It should be noted that while we referred to the two strands being hybridized, not 100% of the bases form base pairing as there are some bases that are mismatched. Also, because rU does not form base pairing with rI as well as rC form base paring with rI, rU provides a focus of hydrodynamic instability in rIₙ.r(C₁₂U)ₙ at the locations of the U bases.

As another example, the formula “rIₙ.r(C₁₁₋₁₄U)ₙ” refers to the same dsRNA except that a ratio of C bases to U bases one strand is about 11 to about 14. That is, the ratio can be 11, 12, 13 or 14 or any value between 11 and 14. For example, when half of the strands are r(C₁₂U)ₙ and half of the strands are r(C₁₃U)ₙ, the formula would be r(C_{12.5}U)ₙ.

The dsRNA (tdsRNA) and ssRNA of this disclosure are homopolymers (e.g., a single-stranded RNA where every base is the same) or heteropolymers (e.g., a single-stranded RNA where the bases can be different) of limited base composition. The tdsRNAs are not mRNA and are distinct from mRNA in structure. For example, the ssRNA and dsRNA are preferably missing one or all of the following: (1) 5′ cap addition, (2) polyadenylation, (3) start codon, (4) stop codon, heterogeneous protein-coding sequences, and (5) spice signals.

As used herein, the term “substantially free” is used operationally in the context of analytical testing of the material. Preferably, purified material is substantially free of one or more impurities. In a preferred embodiment, the tdsRNA of this disclosure is substantially free (e.g., more than 0% to less than 0.1%) or completely free (0%) of dl/dl dsRNA or dCdU/dCdU dsRNA. In other words, the tdsRNA is substantially free or completely free (0%) of homodimers of polymer 1 or homodimers of polymer 2. Substantially free in this context would be considered to be more than 0% but less than 1%, less than 0.5%, less than 0.2%, less than 0.1%, or less than 0.01% of a contaminant such as (1) dl/dl (polymer 1/polymer 1) dsRNA, dCdU/dCdU (polymer 2/polymer 2) dsRNA.

Intravenous (i.v., iv, I.V., or IV) administration refers to administering directly to the vein of a subject with a needle, a tube, a line, a central venous catheter, a peripherally inserted central catheter, tunneled catheter, or an implanted port. IV may be performed by IV push or by infusion. Infusion may be by pump infusion or drip infusion.

Active ingredients or active agents are used interchangeably and include any active ingredient or active agent described in this disclosure including, at least, tdsRNA.

The double-stranded RNAs described in this disclosure are therapeutic double-stranded RNA, abbreviated as “tdsRNA.” tdsRNA includes, at least, Rintatolimod which is a tdsRNA of the formula rIₙ.r(C₁₂U)ₙ). tdsRNA may be stored or administered in a pharmaceutically acceptable solution such as Phosphate Buffered Saline (PBS).

The tdsRNA may be a tdsRNA produced by any of the methods of this disclosure—referred to herein as the “tdsRNA Product” or “tdsRNA”—the two terms have the same meaning. tdsRNA can be represented by one or more of the formulas below in any combination:

rIₙ.r(CₓU)ₙ (formula 1)

rIₙ.r(CₓG)ₙ (formula 2)

rAₙ.rUₙ (also called polyA.polyU) (formula 3)

rIₙ.rCₙ (formula 4)

rugged dsRNA (formula 5)

Each will be discussed further below.

In some embodiments, the tdsRNA may be represented by one or more of the formulas as follows:

rIₙ.r(CₓU)ₙ (formula 1)

rIₙ.r(CₓG)ₙ (formula 2)

In any embodiment, x may be at least one selected from the group consisting of: 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 4-29 (4 to 29), 4-30 (4 to 30), 4-35 (4 to 35), 11-14 (11 to 14), 30-35 (30 to 35). Of these, x=12, and x=11-14 (x may be any value between 11 to 14) are especially preferred.

In these formulas 1 to 5, and in other formulas, where there is no subscript next to a base, the default value is “1.” For example, in the formula rIₙ.r(C₁₂U)ₙ, there is no subscript following “U,” it is understood that rIₙ.r(C₁₂U)ₙ is the same as rIₙ.r(C₁₂U₁)ₙ and the formula is meant to convey that for the strand denoted as r(C₁₂U₁)ₙ, there are 12 rC base for every rU base. Thus, x is also a ratio of the bases of one strand of the tdsRNA. The length of the tdsRNA strand is denoted as a lowercase “n” (e.g., rIₙ.r(C₁₂U)ₙ). The subscript n is also the length of each individual single-stranded nucleic acid. Since tdsRNA is double-stranded, n is also the length of the double-stranded nucleic acid—i.e., the length of the tdsRNA. For example, rIₙ.r(C₁₂U)ₙ indicates, inter alia, a double-stranded RNA with each strand with a length of n.

In another aspect, the tdsRNA may have a formula as follows:

rAₙ.rUₙ (also called polyA.polyU) (formula 3)

rIₙ.rCₙ (formula 4)

In another aspect, the tdsRNA may be a rugged dsRNA (formula 5).

In one embodiment, tdsRNA is at least one selected from the group consisting of formula 1, formula 2, formula 3, formula 4, and formula 5. In another embodiment, tdsRNA comprises formula 1 and formula 2 only. In one preferred embodiment, tdsRNA comprises formula 1 only. In another embodiment, tdsRNA comprises formula 1 and formula 5 (rugged dsRNA).

In another aspect, at least 70%, at least 80%, or at least 90% of the tdsRNA may have a molecular weight of between 400,000 Daltons to 2,500,000 Daltons. Where the term percent (“%”) is used, the percent may be weight percent or molar percent.

In another aspect, the tdsRNA comprises a first ssRNA and a second ssRNA and each of these first ssRNA or second ssRNA may contain one or more strand breaks.

In another aspect, the tdsRNA has the property that greater than about 90%, greater than 95%, greater than 98%, greater than 99%, or 100% of the bases of the RNA are in a double-stranded configuration.

In any aspect, the tdsRNA may be in a therapeutic composition comprising, for example, a tdsRNA, and a pharmaceutically acceptable excipient (carrier).

One embodiment of tdsRNA is directed to rintatolimod, which is a tdsRNA of the formula rIₙ.r(C₁₂U)ₙ and which is also denoted by the trademark AMPLIGEN®.

In a preferred embodiment, the tdsRNA are of the general formula rIₙ.r(C₁₁₋₁₄, U)ₙ and are described in U.S. Pat. Nos. 4,024,222 and 4,130,641 (which are incorporated by reference herein) or synthesized according to this disclosure.

In the case where the tdsRNA is rAₙ.rUₙ, the tdsRNA may be matched (i.e., not in mismatched form).

tdsRNA (e.g., Rintatolimod) has undergone extensive clinical and preclinical testing. It has been well-tolerated in clinical trials enrolling over 1,200 patients with over 100,000 doses administered and there have been no drug-related deaths. Two placebo-controlled, randomized studies show no increase in serious adverse events compared to placebo. Favorable safety profiles have been seen for intraperitoneal, intravenous, and intranasal routes of administration of tdsRNA.

3.1 Length of tdsRNA

The length of the tdsRNA, may be represented by bases for one strand of the tdsRNA or in basepairs for both strands for the tdsRNA. It is understood that in some embodiments that not all of the bases (e.g., U and I) are in basepaired configuration. For example, rU bases do not pair as well as rC bases to inosine.

The length of the tdsRNA may be measured by (1) bases or basepairs, (2) molecular weight which is the weight of the double-stranded tdsRNA (e.g., Daltons) or (3) turns of the double-stranded RNA. These measurements can be easily interconverted. For example, it is generally accepted that there are about 629 Daltons per base pair.

“n” represents length in units of basepair or basepairs (abbreviated as bp regardless of whether it is singular or plural) for double-stranded nucleic acid. “n” can also represent bases for single-stranded RNA. Because “bp” represents singular or plural, it is the same as “bps” which is another representation of basepairs.

The tdsRNA can have the following values for its length “n” (in bases for single strand or basepairs for double strands): 4-5000, 10-50, 10-500, 10-40,000, 40-40,000, 40-50,000, 40-500, 50-500, 100-500, 380-450, 400-430, 400-800 or a combination thereof. Expressed in molecular weight, the tdsRNA may have the following values: 30 kDa to 300 kDa, 250 kDa to 320 kDa, 270 kDa to 300 kDa or a combination thereof. Expressed in helical turns, the tdsRNA may have 4.7 to 46.7 helical turns of duplexed RNA, 30 to 38 helical turns of duplexed RNA, 32 to 36 helical turns of duplexed RNA or a combination thereof.

The length may be an average basepair, average molecular weight, or an average helical turns of duplexed RNA and can take on integer or fractional values.

3.2 Rugged dsRNA (a Form of tdsRNA)

Rugged dsRNA is a tdsRNA that is resistant to denaturation under conditions that are able to separate hybridized poly(riboinosinic acid) and poly(ribocytosinic acid) strands (that is, rIₙ.rCₙ strands). See, U.S. Pat. Nos. 8,722,874 and 9,315,538 (incorporated by reference) for a further description of Rugged dsRNA and exemplary methods of preparing such molecules.

In one aspect, a rugged dsRNA can be an isolated double-stranded ribonucleic acid (dsRNA) which is resistant to denaturation under conditions that are able to separate hybridized poly(riboinosinic acid) and poly(ribocytosinic acid) strands, wherein only a single strand of said isolated dsRNA comprises one or more uracil or guanine bases that are not base-paired to an opposite strand and wherein said single strand is comprised of poly(ribocytosinic₃₀₋₃₅uracilic acid). Further, the single strand may be partially hybridized to an opposite strand comprised of poly(riboinosinic acid). In another aspect, rugged dsRNA may be an isolated double-stranded ribonucleic acid (dsRNA) which is resistant to denaturation under conditions that are able to separate hybridized poly(riboinosinic acid) and poly(ribocytosinic acid) strands.

In another aspect, Rugged dsRNA, has at least one of the following: r(Iₙ).r(C₄₋₂₉U)ₙ, r(Iₙ).r(C₁₂U)ₙ, r(Iₙ).r(C₁₁₋₁₄U)ₙ, r(Iₙ).r(C₃₀U)ₙ, or r(Iₙ).r(C₃₀₋₃₅U)ₙ. In another aspect, Rugged dsRNA may have a size of 4 bps to 5000 bps, 40 bps to 500 bps, 50 bps to 500 bps, 380 bps to 450 bps, 400 bps to 430 bps, 30 kDa to 300 kDa molecular weight, 250 kDa to 320 kDa molecular weight, 270 kDa to 300 kDa molecular weight, 4.7 to 46.7 helical turns of duplexed RNA, 30 to 38 helical turns of duplexed RNA, 32 to 36 helical turns of duplexed RNA, and a combination thereof.

In another aspect, Rugged dsRNA is produced by isolating the 5-minute HPLC peak of a tdsRNA preparation.

3.3 Rugged dsRNA Preparation

In one embodiment, the starting material for making Rugged dsRNA may be dsRNA prepared in vitro using conditions of this disclosure. For example, the specifically configured dsRNA described in U.S. Pat. Nos. 4,024,222, 4,130,641, and 5,258,369 (which are incorporated by reference herein) are generally suitable as starting materials after selection for rugged dsRNA. tdsRNA (or preparations of tdsRNA) described in this disclosure is also useful as starting material.

After procuring starting material, Rugged dsRNA may be isolated by at least subjecting the partially hybridized strands of a population of dsRNA to conditions that denature most dsRNA (more than 10 wt % or mol %, more than 20 wt % or mol %, more than 30 wt % or mol %, more than 40 wt % or mol %, more than 50 wt % or mol %, more than 60 wt % or mol %, more than 70 wt % or mol %, more than 80 wt % or mol %, more than 90 wt % or mol %, more than 95 wt % or mol %, or more than 98 wt % or mol %) in the population, and then selection negatively or positively (or both) for dsRNA that remain partially hybridized. The denaturing conditions to unfold at least partially hybridized strands of dsRNA may comprise an appropriate choice of buffer salts, pH, solvent, temperature, or any combination thereof. Conditions may be empirically determined by observation of the unfolding or melting of the duplex strands of ribonucleic acid. The yield of rugged dsRNA may be improved by partial hydrolysis of longer strands of ribonucleic acid, then selection of (partially) hybridized stands of appropriate size and resistance to denaturation.

The purity of rugged dsRNA, which functions as tdsRNA, may thus be increased from less than about 0.1-10 mol % (e.g., rugged dsRNA is present in at least 0.1 mol % or 0.1 wt percent but less than about 10 mol % or 10 wt percent) relative to all RNA in the population after synthesis to a higher purity. A higher purity may be more than 20 wt % or mol %, more than 30 wt % or mol %, more than 40 wt % or mol %, more than 50 wt % or mol %, more than 60 wt % or mol %, more than 70 wt % or mol %, more than 80 wt % or mol %, more than 90 wt % or mol %, more than 98 wt % or mol %, or between 80 to 98 wt % or mol %. All wt % or mol % is relative to all RNA present in the same composition.

Another method of isolating Rugged dsRNA is to employ chromatography. Under analytical or preparative high-performance liquid chromatography, Rugged dsRNA can be isolated from a preparation (e.g., the starting material as described above) to produce poly(I):poly(C₁₂U), (e.g., poly(I):poly(C₁₁₋₁₄U)ₙ) as a substantially purified and pharmaceutically-active molecule with an HPLC peak of about 4.5 to 6.5 minutes, preferably between 4.5 and 6 minutes and most preferably 5 minutes.

Rugged dsRNA and the method of making rugged dsRNA are described in U.S. Pat. Nos. 8,722,874 and 9,315,538 (incorporated by reference).

3.4 Stabilizing Polymers

In any of the described embodiments, the tdsRNA may be complexed with a stabilizing polymer such as: polylysine, polylysine plus carboxymethylcellulose (lysine carboxy methyl cellulose), polyarginine, polyarginine plus carboxymethylcellulose, or a combination thereof. Some of these stabilizing polymers are described, for example, in U.S. Pat. No. 7,439,349.

3.5 Modified Backbone

The tdsRNA may comprise one or more alterations in the backbone of the nucleic acid. For example, configured tdsRNA may be made by modifying the ribosyl backbone of poly(riboinosinic acid) r(Iₙ), for example, by including 2′-O-methylribosyl residues. Specifically configured dsRNA may also be modified at the molecule's ends to add a hinge(s) to prevent slippage of the base pairs, thereby conferring specific bioactivity in solvents or aqueous environments that exist in human biological fluids.

4. Administration (Delivery)

Administration to the subject or administering to the subject of any composition or medicament of this disclosure may be in any known form including: systemic administration; parenteral administration (e.g., subcutaneous, intravenous, intramuscular, intradermal, or intraperitoneal; buccal, sublingual, transmucosal; inhalation, instillation intranasally or intratracheally); intradermal administration; subcutaneous administration; intramuscular administration; intranasal administration (pulmonary airway administration); intranasal and oral administration; intraperitoneal administration; intracranial administration; intravesical administration; oral administration (through the mouth, by breathing through the mouth); topical administration (e.g., device such as a nebulizer for inhalation through the respiratory system, skin patch acting epicutaneously or transdermally, suppository acting in the rectum or vagina). One especially preferred administration method is intravenous administration.

5. Formulations and Dosage

Formulations for administration (i.e., pharmaceutical compositions) may include a pharmaceutically acceptable carrier with the tdsRNA.

Pharmaceutical carriers include suitable non-toxic vehicles in which a composition of the disclosure is dissolved, dispersed, impregnated, or suspended, such as water or other solvents, fatty materials, celluloses and their derivatives, proteins and their derivatives, collagens, gelatine, polymers, adhesives, sponges, fabrics, and the like and excipients which are added to provide better solubility or dispersion of the drug in the vehicle. Such excipients may include non-toxic surfactants, solubilizers, emulsifiers, chelating agents, binding materials, lubricants, softening agents, and the like. Pharmaceutically acceptable carriers may be, for example, aqueous solutions, syrups, elixirs, powders, granules, tablets, and capsules which typically contain conventional excipients such as binding agents, fillers, lubricants, disintegrants, wetting agents, suspending agents, emulsifying agents, preservatives, buffer salts, flavoring, coloring, and/or sweetening agents.

The tdsRNA may be a combination or any subset of dsRNA described above (e.g., formula (1) to formula (5)). It is understood that in one aspect, tdsRNA may comprise a combination of all of the examples of tdsRNA described above or any subset of the above examples. With respect to the subsets, the specific exclusion of one or more specific embodiments of tdsRNA is also envisioned. As nonlimiting examples, tdsRNA may comprise any of the following: (A) all of the examples of tdsRNA as described above; (B) all of the examples of tdsRNA described above but without rIₙ.r(C₁₁₋₁₄U)ₙ; (C) Rugged dsRNA; (D) rIₙ.r(C₁₂U)ₙ; (E) tdsRNA as described above but without rIₙ.r(C₁₁₋₁₄U)ₙ and without Rugged dsRNA; (F) rIₙ.r(C₁₂U)ₙ, and Rugged dsRNA; or (G) rIₙ.r(C₁₁₋₁₄U)ₙ and Rugged dsRNA.

5.1 Medicament

In another aspect, a medicament (e.g., a pharmaceutical composition) containing the tdsRNA is provided. Optional other components of the medicament include excipients and a vehicle (e.g., aqueous buffer or water for injection) packaged aseptically in one or more separate containers (e.g., nasal applicator or injection vial). Further aspects will be apparent from the disclosure and claims herein.

5.2 Dosage for the Average Subject

The dosages are generally applicable to a subject as described in another section of this disclosure. In a preferred embodiment, the subject is human.

For a subject (especially human) the dose of tdsRNA for iv administration may be: 0.1 μg to 1,200 mg; 0.1 to 25 mg; 25 mg to 50 mg; 50 mg to 100 mg; 100 mg to 200 mg; 200 mg to 400 mg; 400 mg to 800 mg; 800 mg to 1,200 mg. For example, iv dosages may be 25 mg; 50 mg; 125 mg; 250 mg; 500 mg; 1,000 mg; 1,200 mg.

For intranasal dosage, the dose of tdsRNA may be: 0.1 μg to 1,200 μg; 0.1 to 25 μg; 25 μg to 50 μg; 50 μg to 100 μg; 100 μg to 200 μg; 200 μg to 400 μg; 400 μg to 800 μg; 800 μg to 1,250 μg. For example, intranasal dosages may be 25 μg; 50 μg; 125 μg; 250 μg; 500 μg; 1,000 μg; 1,250 μg.

5.3 Amount Per Unit Dose

The amount per unit dose of tdsRNA may be at least one selected from 0.1 mg/kg, 0.2 mg/kg, 0.4 mg/kg, 0.6 mg/kg, 0.8 mg/kg, 1 mg/kg, 2 mg/kg, 3 mg/kg, 4 mg/kg, 5 mg/kg, 6 mg/kg, 8 mg/kg, 10 mg/kg.

5.4 Specific Examples

In one embodiment, the tdsRNA is administered iv at a dose from about 1 mg/kg to 10 mg/kg biweekly. As another example, the administration may be in 50-1400 milligrams every other day, leading to an average daily dosage of 25-700 milligrams per day. In one embodiment, the tdsRNA is administered at a dose from about 0.50 mg/kg to 10 mg/kg every other week. 50-1400 milligrams every other day, leading to an average daily dosage of 25-700 milligrams per day.

One preferred dosage is tdsRNA (e.g., rintatolimod (AMPLIGEN®)) 200 mg twice weekly for 2 weeks, then 400 mg twice weekly. The treatment may be maintained for 18 weeks or be continuous. Continuous treatment, in this case, refers to 400 mg twice weekly after the initial 2 weeks. A preferred method of administration is intravenous administration.

5.5 Dose Frequency

In certain embodiments, the tdsRNA is administered at a frequency selected from the group consisting of: one dose per day, one dose every 2 days, one dose every 3 days, one dose every 4 days, one dose every 5 days, 4 doses a week, 3 doses a week, 2 doses a week, 1 dose a week, one dose every two weeks, one dose every three weeks, one dose every four weeks, and one dose every month. Nasal administration may be as listed above or may be 2 doses per day or three doses per day. Administration or dosing can be continued as long as they have a beneficial effect on the subject.

6. Discussion of Further Embodiments and Features

The safety and efficacy of tdsRNA has been extensively tested. Ampligen® (rintatolimod) is an optimized TLR3 agonist and endogenous interferon (IFN) inducer in late-stage clinical development with the ability to augment both innate and acquired immunity including cellular responses (T-cells) in humans with immunodeficiency (HIV disease) (Thompson et al., 1996), as well as, NK cells and humoral (B-cells) responses, in normal human volunteers (Overton et al., 2014; Zarling et al. 1980; Strayer et al. 2015).

Ampligen® has undergone extensive preclinical safety testing including 18 animal (mice, rat, rabbit, dog, and monkey) toxicity studies; 6 animal (rat and rabbit) reproductive studies, and 2 animal (rat, monkey) PK/PD studies. A comparison of Human vs. NHP PK parameters for a dose of 6 mg/kg is shown in the TABLE below. The MTD for humans and NHPs is >10 mg/kg and >36 mg/kg, respectively.

TABLE 3
PK Parameters Comparing Humans to NHP Receiving Ampligen ®
by IV Infusion Twice Weekly Over 30 Minutes

| | | | AUC ¹ μg- | |
|---|---|---|---|---|
| | Half-Life ¹ (min) | Cmax ¹ (μg/ml) | in/ml) | MTD (mg/kg) |
| Humans | 36.5 | 74.0 | 5,220 | >10²→17³ |
| NHP⁶ | 22.9 | 22.4 | 10,260 | >36⁴-100⁵ |

¹ Based on 6 mg/kg infused over 30 minutes with PK sampling initiated 10 minutes after end of infusion,
² MTD based on randomized, well-controlled 24 week study in HIV disease,
³ MTD based on 1200 mg BIW (cancer),
⁴ MTD based on 24 weeks,
⁵ MTD based on 4 weeks,
⁶Cynomolgus monkey.

7. Discussion of Further Embodiments and Features

7.1 Subject or Patient

As used herein, a “subject” has the same meaning as a “patient” and is a mammal, preferably a human. In addition to humans, categories of mammals within the scope of the present disclosure include, for example, farm animals, domestic animals, laboratory animals, etc. Some examples of farm animals include cows, pigs, horses, goats, etc. Some examples of domestic animals include dogs, cats, etc. Some examples of laboratory animals include primates, rats, mice, rabbits, guinea pigs, etc. Other examples of subjects include any animal such as civet cats, swine, cattle, horses, camels, cats, dogs, rodents, birds, bats, rabbits, ferrets, mink, snake, and the like. In this disclosure, the terms “patient” and “subject” are used interchangeably. 7.2 Effective Amount: Therapeutically or Prophylactically Effective Amount

The compositions are delivered in effective amounts. The term “effective amount” refers to the amount necessary or sufficient to realize a desired biological effect which is, for example, reducing, stopping the advance of, or reversing the symptoms of cancer. In addition to the sample dosages and administration methods mentioned, one of ordinary skill in the art can empirically determine the effective amount of the tdsRNA without necessitating undue experimentation. It is preferred that a maximum dose be used, that is, the highest safe dose according to medical judgment.

Effective dosage forms, modes of administration, and dosage amounts may be determined empirically, and making such determinations is within the skill of the art. It is understood by those skilled in the art that the dosage amount will vary with the route and mode of administration, the rate of excretion, the duration of the treatment, the identity of any other drugs (e.g., antiviral agent) being co-administered, the age, size, species of mammal (e.g., human patient), and other factors well known in the arts of medicine and veterinary medicine. In general, a suitable dose of any active agent disclosed herein or a composition containing the same will be that amount of the active agent (tdsRNA) or composition comprising the active agent, which is the lowest dose effective to produce the desired effect. The desired effect may be to reduce the severity or duration of a symptom of cancer.

8. Other Aspects

In this specification, stating a numerical range, it should be understood that all values within the range are also described (e.g., one to ten also includes every value between one and ten as well as all intermediate ranges such as two to ten, one to five, and three to eight). The term “about” may refer to the statistical uncertainty associated with a measurement or the variability in a numerical quantity that a person skilled in the art would understand does not affect the operation of the disclosure or its patentability.

All modifications and substitutions that come within the meaning of the claims and the range of their legal equivalents are to be embraced within their scope. A claim which recites “comprising” allows the inclusion of other elements to be within the scope of the claim. The disclosure is also described by such claims reciting the transitional phrases “consisting essentially of” (i.e., allowing the inclusion of other elements to be within the scope of the claim if they do not materially affect the operation of the disclosure) or “consisting of” (i.e., allowing only the elements listed in the claim other than impurities or inconsequential activities which are ordinarily associated with the disclosure) instead of the “comprising” term. While the three transitions “comprising,” “consisting of,” and “consisting essentially of” have different meanings, they can be substituted for each other whenever used to create new embodiments of the disclosure.

An element described in this specification should not be construed as a limitation of the claimed disclosure unless it is explicitly recited in the claims. Thus, the granted claims are the basis for determining the scope of legal protection instead of a limitation from the specification which is read into the claims. In contradistinction, the prior art is explicitly excluded from the disclosure to the extent of specific embodiments that would anticipate the claimed disclosure or destroy novelty.

Moreover, no particular relationship between or among limitations of a claim is intended unless such relationship is explicitly recited in the claim (e.g., the arrangement of components in a product claim or order of steps in a method claim is not a limitation of the claim unless explicitly stated to be so). All possible combinations and permutations of individual elements, embodiments, and aspects disclosed herein are also considered to be aspects and embodiments of the disclosure. Similarly, generalizations of the disclosure's description are considered to be part of the disclosure.

From the foregoing, it would be apparent to a person of skill in this art that the disclosure can be embodied in other specific forms without departing from its spirit or essential characteristics.

While the disclosure has been described in connection with what is presently considered to be the most practical and preferred embodiment, it is to be understood that the disclosure is not to be limited to the disclosed embodiment, but on the contrary, is intended to cover various modifications and equivalent arrangements included within the spirit and scope of the appended claims.

9. Incorporation by Reference

All publications, patent applications, and patents mentioned herein are hereby incorporated by reference in their entirety as if each individual publication or patent was specifically and individually indicated to be incorporated by reference. These patents include, at least, U.S. Pat. Nos. 4,024,222, 4,130,641, 5,258,369, 7,439,349, 8,722,874 and 9,315,538. In case of conflict, the present application, including any definitions herein, will control.

### EXAMPLES

### Example 1 Testing the Effects of a Combination of FOLFIRINOX and Rintatolimod on Pancreatic Cancer

A program was conducted to see if tdsRNA in combination with FOLFIRINOX would be more effective in treating pancreatic cancer than the current standard of care—which is a FOLFIRINOX regimen. In this case, the tdsRNA used is rintatolimod (AMPLIGEN®) which is a form of tdsRNA. Included in the study were adults with metastatic or locally advanced pancreatic carcinoma following FOLFIRINOX treatment. The tdsRNA treatment was rintatolimod (AMPLIGEN®) administered i.v. 200 mg twice weekly for 2 weeks, then 400 mg twice weekly for a total treatment duration of 18 weeks.

The experiment compared survival in the experimental AMPLIGEN® cohort (n=27) compared to a historical control group (n=27) matched for age, gender, disease stage and number of cycles of FOLFIRINOX. As shown in Table 4, the experimental and the control groups were well-matched.

To be selected for the study, the patient should be over 18 years in age and diagnosed with locally advanced pancreatic cancer (LAPC) or metastasized pancreatic cancer. These LACP patients were not candidates for surgical resection. The patient should have completed the standard of care (e.g., A FOLFIRINOX regimen) and the patient should have no progressive disease (as detected by computed tomography (CT) 6 weeks after treatment). Patients with second malignancy (simultaneous), on immunosuppressive medication, or having liver-renal insufficiency were excluded.

Survival analysis was performed as follows. A group of 136 patients was assembled from a retrospective database, including all patients who received FOLFIRINOX between 2012-2018. Variables that were matched include age (+ or −10 years); gender; disease stage (locally advanced pancreatic cancer (LAPC), metastasized, metastasized after resection); and the number of FOLFIRINOX cycles to obtain a historical control group that was well-matched to the experimental group. The outcomes were determined from the start of FOLFIRINOX, and the overall survival and progression-free survival were calculated.

TABLE 4
Patient Characteristics of Control Group Compared to
Experimental AMPLIGEN ® Group

| | | Experimental | |
|---|---|---|---|
| | | AMPLIGEN ® | |
| | Control Group | Group | |
| Variable | (n = 27) | (n = 27) | p-value* |
| Age, mean (sd) | 64.0 ± 8.4 | 62.7 ± 8.4 | 0.563 |
| FOLFIRINOX cycles, | 7.70 ± 3.7 | 8.04 ± 3.3 | 0.727 |
| mean (sd) | | | |
| | | Experimental | |
| | | AMPLIGEN ® | |
| Gender | Control Group | Group | p-value** |
| Male (n) | 18 | 19 | 0.770 |
| Female (n) | 9 | 8 | |
| Male (%) | 67% | 70% | |
| Female (%) | 33% | 30% | |
| | | Experimental | |
| | | AMPLIGEN ® | |
| Disease Stages | Control Group | Group | p-value |
| LAP | | | N/a*** |
| CN | 5 | 5 | |
| % | 19% | 19% | |
| Metastatic | | | N/a*** |
| N | 22 | 22 | |
| % | 81% | 81% | |

*Student's T-test (Two-tailed)
**Chi-squared Test (Two-tailed)
***N/a = not applicable since percentages were identical in the two groups
Control Group B was also well-matched with regard to Age, Cycles of FOLFIRINOX, Gender, and Disease Stages.

TABLE 5
A Comparison of Overall Survival Shows a Statistically
Significant Increase in the Experimental AMPLIGEN ®
Group Compared to the Control Group

| | Experimental AMPLIGEN ® | |
|---|---|---|
| Control Group (n = 27) | Group (n = 27) | |
| Median Overall Survival | Median Overal | |
| (months) | lSurvival (months) | p-value* |
| 12.0 | 19.0 | 0.035 |

*Mann-Whitney U Test (Two-tailed)

TABLE 6
A Comparison of Progression-free Survival Shows a Statistically
Significant Increase in the Experimental AMPLIGEN ®
Group ompared to Control Group

| | Experimental AMPLIGEN ® | |
|---|---|---|
| Control Group (n = 27) | Group (n = 27) | |
| Median Progression-free | Median Progression-free | |
| Survival (months) | Survival (months) | p-value* |
| 8.0 | 12.0 | 0.012 |

*Mann-Whitney U Test (Two-tailed)

Based on the data, we can conclude that AMPLIGEN® treatment of pancreatic cancer following FOLFIRINOX yielded a significant additional 12.0 months of increased overall survival (OS) compared to the control group. AMPLIGEN® treatment in an EAP of pancreatic ductal adenocarcinoma following FOLFIRINOX yielded significant (p<0.035) progression-free survival and overall survival benefits compared to matched historical controls. The data show a median (50%) overall survival (OS) of 12.0 months in controls vs. 19.0 months in the AMPLIGEN® following FOLFIRINOX cohort which was statistically and medically significantly different (see TABLE 6). Also, median (50%) progression-free survival (PFS) of 8.0 months in controls vs. 12.0 months in AMPLIGEN® following FOLFIRINOX cohort was also statistically and medically significantly different (see TABLE 7).

The magnitude of the AMPLIGEN® survival benefit of 7.0 (19.0-12.0) months is highly clinically significant compared to the other forms of therapy available for advanced pancreatic ductal carcinoma. Comparison of the overall survival (OS) benefits from using AMPLIGEN® following FOLFIRINOX (which added 7.0 months for a total of 19.0 months of OS) to the OS benefit obtained from using the drugs approved for pancreatic adenocarcinoma is shown in TABLE 7.

Based on the results, the combination of FOLFIRINOX followed by AMPLIGEN® yielded a remarkable overall survival (OS) benefit of 19 months. Moreover, the AMPLIGEN® therapy was for only 18 weeks of twice weekly infusions, which are generally well-tolerated.

Our analysis and previous studies have shown that AMPLIGEN® has a very good safety profile. AMPLIGEN® lacks the potent toxicities seen with most chemotherapeutic agents. See, e.g., AMPLIGEN®'s safety profile in other parts of this disclosure. There is no evidence of any cumulative toxicities, and AMPLIGEN® can be used as long as the patients are benefitting. Thus, we believe the ideal use of AMPLIGEN® would be to continue therapy beyond 18 weeks until time of tumor progression. We believe this approach could add an additional 4 to 6 months or more to the overall survival (OS) benefit.

In conclusion, this study showed that the twenty-seven patients with advanced pancreatic cancer treated with FOLFIRINOX followed by AMPLIGEN® had an overall survival of 19 months. This represents a 7.0 month increased survival benefit compared to the current standard of Care (SOC) using FOLFIRINOX. In addition, compared to the control group, AMPLIGEN®'s overall survival benefit of 19 months was 7.0 months longer than the Control Group (TABLE 5).

Table 7 shows that the highest overall survival (OS) benefit from drugs approved is 11.1 months (FOLFIRINOX followed by Gemcitabine). Next is Abraxane plus Gemcitabine which yielded an OS benefit of 8.5 months. The only other therapy with an OS greater than 7 months is Lynparza, which is approved for a small subset of patients (approximately 5-7%) with metastatic adenocarcinoma and a germline BRCA-mutation. Therefore, 93-95% of patients with metastatic adenocarcinoma of the pancreas are not eligible to receive Lynparza. The other approved combinations, Tarceva plus Gemcitabine and Onivyde plus 5-FU/Leucovorin have OS benefits of 6.5 and 6.1 months, respectively.

Thus, the combination of FOLFIRINOX followed by Ampligen yielded a remarkable overall survival (OS) benefit of 19 months. Moreover, the Ampligen therapy was for only 18 weeks of twice-weekly infusions, which are generally well-tolerated (see FIG. 1 and Table 12). Continuing Ampligen therapy until time of progression would be expected to increase the overall survival benefit.

TABLE 7
Summary of Median Overall Survival (OS) Benefits from Approved
Drugs or Drug Combinations for Pancreatic Adenocarcinoma

| First-Line Therapy | OS Months | OS Over Control |
|---|---|---|
| 1. FOLFIRINOX followed by | 11.1 | 4.3 |
| gemcitabine vs. gemcitabine alone | 6.8 | |
| 2. Abraxane + gemcitabine | 8.5 | 1.8 |
| vs. gemcitabine alone | 6.7 | |
| 3. Tarceva + gemcitabine | 6.5 | 0.5 |
| vs. gemcitabine alone | 6.0 | |
| Second-Line Therapy | OS Months | OS Over Control |
| 4. Lynparza monotherapy* | 7.4 | 3.6 |
| vs. placebo | 3.8 | |
| 5. Onivyde + 5-FU/Leucovorin** | 6.1 | 1.9 |
| vs. 5-FU/Leucovorin alone | 4.2 | |

*For germline BRCA-mutated metastatic adenocarcinoma and have not progressed after at least 16 weeks of platinum used in a first-line chemotherapy regimen. Only 5-7% of patients with metastaticpancreatic adenocarcinoma have a germline BRCE mutation.
**Following Gemcitabine based first-line therapy.

1. A method for treating a cancer in a subject in need thereof, the method comprising:
a first step of administering to the subject a cancer therapy which is a standard of care for the cancer; and
a second step of administering to the subject at least an effective amount of a therapeutic double stranded RNA (tdsRNA).

2. A method for treating a cancer in a subject in need thereof, the method comprising:
administering to the subject a compound comprising an effective amount of a therapeutic double stranded RNA (tdsRNA).

3. The method of claim 1, wherein the cancer is pancreatic cancer.

4. The method of claim 3, wherein the pancreatic cancer is at least one selected from the group consisting of:
pancreatic carcinoma;
advanced pancreatic carcinoma;
locally advanced pancreatic cancer (LAPC);
metastasized pancreatic cancer; and
pancreatic cancer metastasized after resection.

5. The method of claim 1, wherein the standard of care is a FOLFIRINOX treatment regimen, and wherein the first step is administering to the subject the FOLFIRINOX treatment regimen.

6. The method of claim 1, wherein the standard of care is a Gemcitabine treatment regimen, and wherein the first step is administering to the subject the Gemcitabine treatment regimen.

7. The method of claim 1, wherein the first step and the second step are performed in any order or simultaneously.

8. The method of claim 1, wherein the tdsRNA is at least one selected from the group consisting of
rIₙ.r(CₓU)ₙ (formula 1)
rIₙ.r(CₓG)ₙ (formula 2)
rAₙ.rUₙ (also called polyA.polyU) (formula 3)
rIₙ.rCₙ (formula 4)
rugged dsRNA (formula 5)
wherein x is at least one selected from the group consisting of 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 4-29, 4-30, 14-30, 15-30, 11-14, and 30-35.

9. The method of claim 8,
wherein at least 90 wt % of the tdsRNA is larger than 40 basepairs; or
wherein at least 90 wt % of the tdsRNA is smaller than 50,000 basepairs.

10. The method of claim 8, wherein n is a number with a value selected from 40 to 50,000.

11. The method of claim 8,
wherein n is from 40 to 40,000;
wherein the tdsRNA has about 4 to about 4000 helical turns of duplexed RNA strands; or
wherein the tdsRNA has a molecular weight of
2 kDa to 30,000 kDa.

12. The method of claim 8, wherein the tdsRNA comprises
rIₙ.r(C₁₁₋₁₄U)ₙ; and
rugged dsRNA.

13. The method of claim 8,
wherein the rugged dsRNA comprises
a single strand comprised of r(C₄₋₂₉U)ₙ, r(C₁₁₋₁₄U)ₙ, or r(C₁₂U)ₙ; and
an opposite strand comprised of r(I);
wherein the single strand and the opposite strand do not base pair the position of the uracil base, and
wherein the single strand and the opposite strand are partially hybridized.

14. The method of claim 8,
wherein
the rugged dsRNA has a molecular weight of about 250 kDa to 500 kDa;
each strand of the rugged dsRNA is from about 400 to 800 basepairs in length; or
the rugged tdsRNA has about 30 to 100 or 30 to 60 helical turns of duplexed RNA.

15. The method of claim 8, wherein the Rugged dsRNA is resistant to denaturation under conditions that are able to separate hybridized poly(riboinosinic acid) and poly(ribocytosinic acid) strands (rIₙ.rCn).

16. The method of claim 8, wherein the rugged dsRNA is an isolated double-stranded ribonucleic acid (dsRNA) active under thermal stress comprising:
each strand with a molecular weight of about 250 KDa to about 500 KDa, 400-800 basepairs, or 30 to 60 helical turns of duplex RNA;
a single strand comprised of poly(ribocytosinic₄₋₂₉ uracilic acid) and an opposite strand comprised of poly(riboinosinic acid);
wherein the two strands do not base pair the position of the uracil base;
wherein the two strands base pair the position of the cytosine base; and
wherein the strands are partially hybridized.

17. The method of claim 8, wherein the tdsRNA comprises 0.1-12 mol % rugged dsRNA.

18. The method of claim 1, wherein the tdsRNA comprises at least one pharmaceutically acceptable carrier.

19. The method of claim 1, wherein administering the tdsRNA is performed by at least one method selected from the group consisting of:
intravenous administration;
systemic administration;
parenteral administration;
intradermal administration;
subcutaneous administration;
intramuscular administration;
intranasal administration;
intranasal and oral administration;
intraperitoneal administration;
intracranial administration;
intravesical administration;
oral administration;
topical administration; and
enteral administration.

20. The method of claim 1, wherein the tdsRNA is administered at a dosage of 25 mg to 700 mg of tdsRNA per dose.

21. The method of claim 1, wherein the tdsRNA is administered 200 mg twice weekly for two weeks, and 400 mg twice weekly after the first two weeks.

22. The method of claim 1, wherein the tdsRNA is administered at a frequency selected from the group consisting of:
one dose per day,
one dose every 2 days,
one dose every 3 days,
one dose every 4 days,
one dose every 5 days,
one dose a week,
two doses a week,
three doses a week,
one dose every two weeks,
one dose every 3 weeks,
one dose every 4 weeks, and
one dose a month.

23. The method of claim 1, wherein the subject is a mammal, preferably a human.

24. The method of claim 1, wherein the tdsRNA further comprises a pharmaceutically acceptable carrier.

25. The method of claim 1, wherein the method has an effect on the subject which is at least one selected from the group consisting of:
increasing survival of the subject;
increasing time of progression of the subject;
inhibiting tumor growth;
inducing tumor cell death;
increasing tumor regression;
preventing tumor recurrence;
preventing tumor growth;
preventing tumor spread;
delaying tumor recurrence;
delaying tumor growth;
delaying tumor spread; and
promoting tumor elimination.
